(19) United States
(12) Patent Application Publication
(10) Pub. No.: US 2004/0195040 A1
(43) Pub. Date: Oct. 7, 2004

(54) Powerful sound for powerful engines

(76) Inventors: Manish Vaishya, Rochester Hills, MI (US); John R. Mammarella, Windsor (CA); Gregory C. Langford, Washington, MI (US); David Maclay, Lansdale, PA (US)

Correspondence Address: CARLSON, GASKEY & OLDS, P.C., 400 WEST MAPLE ROAD, SUITE 350, BIRMINGHAM, MI, 48009, US

(21) Appl. No.: 10/818,200

(22) Filed: Apr. 5, 2004

Related U.S. Application Data
(60) Provisional application No. 60/460,489, filed on Apr. 4, 2003.

(51) Int. Cl. A61F 11/06; G10K 11/16; H03B 29/00

(52) U.S. Cl. 181/206; 381/71.5

(57) ABSTRACT

An air induction system includes a duct that defines a passage for airflow to the engine. The duct is substantially clear of obstructions to improve airflow that in turn provides improved engine performance. A speaker generates sounds to provide a desirable engine sound. A controller drives the speaker to generate desired tones and sounds responsive to engine operation. The sounds generated by the speaker enhance, improve and provide desirable and proportional acoustic performance more reflective of the improved engine performance provided by the improved airflow.

This application claims priority to U.S. Provisional Application No. 60/460,489, which was filed on Apr. 4, 2003.

### BACKGROUND OF THE INVENTION

This invention relates generally to an air induction system for a vehicle, and specifically to an air induction system providing improved air flow characteristics and acoustical performance.

Typically, an engine includes an air induction system to channel air into an intake manifold for mixing with fuel. Conventional Air induction systems typically include an air box, induction orifice and multiple resonators. Each of these structures restricts airflow and reduces engine performance. It is known to provide an air induction system without the air box, induction orifice or resonators to remove restrictions to airflow that in turn result in improved engine performance and efficiencies.

Disadvantageously, removal of resonators from the air induction system can result in undesirable acoustic performance. Undesirable acoustic performance includes inconsistent sounds that are not indicative of the increased and improved engine performance. Further, removal of resonators from the air induction system can lead to unexpected sounds and sounds that are not proportional to engine operating conditions. Such poor acoustic performance can disrupt and degrade the otherwise desirable improvements in engine performance.

Accordingly, it is desirable to develop an air induction system that improves airflow while providing desirable acoustical performance.

### SUMMARY OF THE INVENTION

This invention is an air induction system that includes a low restriction duct providing improved airflow and a speaker for generating sounds to provide desired acoustic characteristics reflecting the improved engine operation.

The air induction system includes a duct that defines a passage for airflow to the engine. An air filter having improved airflow properties is disposed within the passage to prevent contaminants from being carried into the engine. The air induction system includes a speaker driven by a controller to generate tones responsive to engine operation. The duct is substantially clear of obstructions to airflow to provide improved engine performance. Undesirable sounds caused by the substantially unobstructed airflow through the duct can detract from the improvements in engine performance. For this reason the speaker is driven by the controller to generate tones and sounds that enhance, improve and provide desirable and proportional acoustic performance more reflective of the improved engine performance.

Accordingly, the air induction system of this invention provides improved airflow while providing desirable and proportional acoustical performance reflective of the improved engine performance.

These and other features of the present invention can be best understood from the following specification and drawings, the following of which is a brief description.

### BRIEF DESCRIPTION OF THE DRAWINGS

FIG. 1 is a schematic view of a vehicle including an air induction system according to this invention.

FIG. 2 is a schematic representation of the air induction system according to this invention.

FIG. 3 is a schematic representation of another air induction system according to this invention.

FIG. 4 is a flow diagram illustrating an algorithm for generation of desired sounds according to this invention.

### DETAILED DESCRIPTION OF THE PREFERRED EMBODIMENT

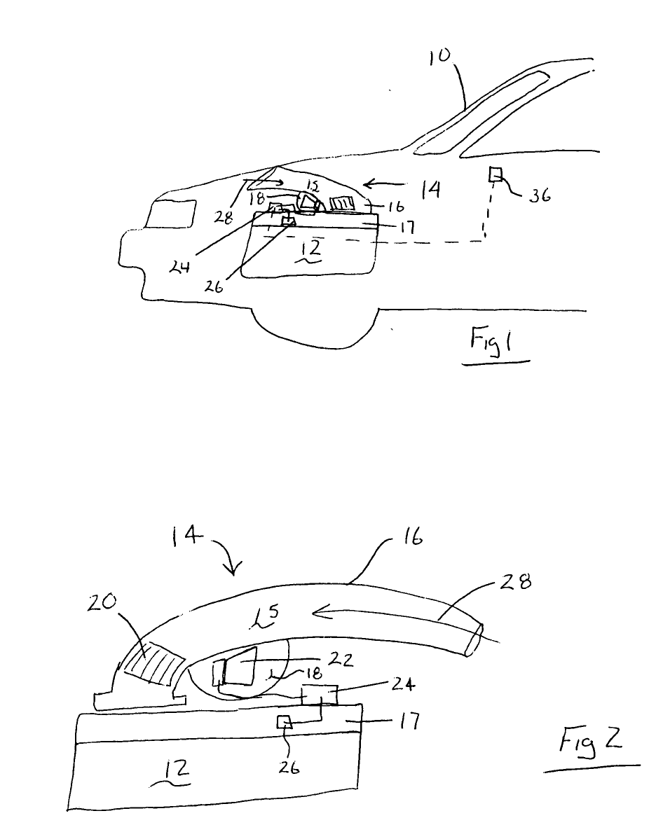

Referring to FIG. 1, a vehicle 10 includes an air induction system 14 for supplying air to an engine 12. The air induction system 14 includes a duct 16 that defines a passage 15 for airflow 28 to an intake manifold 17 of the engine 12. An air filter 20 is disposed within the passage to prevent contaminants from being carried into the engine 12 with the airflow 28. The air induction system 14 includes a controller 24 that drives a speaker 22 to generate tones responsive to engine operation. The tones generated by the speaker 22 along with the sounds generated by the engine 12 provide desirable acoustic properties proportional to engine performance.

Referring to FIG. 2, the duct 16 is substantially clear of obstructions to airflow 28 to provide improved engine performance. Obstructions such as for example, resonators, and an air box or flow restriction that are present in some conventional induction system restrict airflow to the engine 12, thereby limiting engine performance. The duct 16 of this invention provides a substantially unobstructed passage for airflow 28 to improve engine performance.

The air filter 20 is disposed within the passage 15 and provides for the filtering of air. Preferably, the air filter 20 is of a configuration providing minimal resistance to airflow 28. The air filter 20 configuration includes a larger than conventional filter surface area to provide the desired increase in airflow therethrough. The specific configuration of the air filter 20 is as known.

The duct 16 is preferably fabricated from a plastic material. Although plastic material is preferable, the use of other materials such as aluminum or metallic materials is within the contemplation of this invention. Using a plastic material provides the advantage of maintaining the air at a cooler temperature for a longer duration as compared to ducts fabricated from aluminum or metallic materials.

The improved engine performance provided by unobstructed airflow 28 is known to cause undesirable acoustic characteristics. The removal of resonators and other items that obstruct airflow 28 can cause unexpected, unpleasant and undesirable sounds that may not be proportional to current engine performance. The undesirable sounds caused by the substantially unobstructed airflow through the duct 16 can detract from the improvements in engine performance. For this reason the speaker 22 is included for generating tones and sounds that enhance, improve and provide desirable and proportional acoustic performance.

The speaker 22 is supported within a speaker chamber 18. The speaker chamber 18 supports the speaker 22 and provides for amplification of emitted sound to provide the desired tones. The speaker chamber 18 is disposed adjacent the passage 15, but is not in communication with the passage 15. Preferably the speaker chamber 18 is disposed adjacent an end of the duct 16 adjacent the engine 12. Although a preferred location for the speaker chamber 18 is illustrated, a worker with the benefit of this disclosure would understand that the location for the speaker 22 and speaker chamber 18 could vary to provide desired acoustic characteristics.

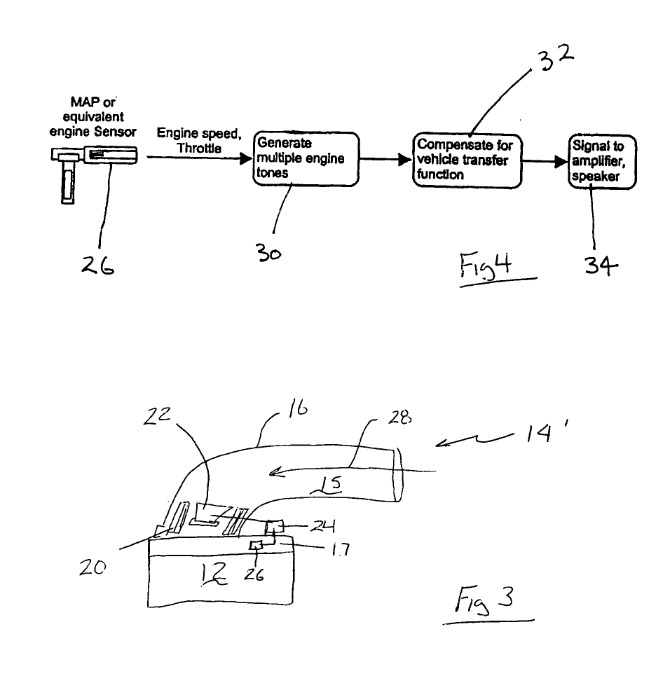

Referring to FIG. 3, an alternate location for the speaker 22 is shown disposed within the duct 16. The speaker 22 is disposed within the air filter 20 to provide a more compact duct 16. As appreciated, a worker with the benefit of this disclosure will recognize that the position of the speaker 22 is application specific and can be changed depending on the desired acoustic characteristics. Further, a worker with the benefit of this disclosure will recognize that the size and power of the speaker 22 can vary depending on the desired sound characteristics.

The controller 24 drives the speaker 22 to generate the desired sound profile. The controller 24 is in communication with a manifold absolute pressure (MAP) sensor 26. The MAP sensor 26 measures pressure within the intake manifold 17 to provide an indication of engine operating conditions, such as the speed and throttle opening. The controller 24 utilizes data indicative of engine speed to adjust the sound emitted by the speaker 22 in order to craft, amplify and condition the overall acoustic characteristic of the engine 12 during operation. The MAP sensor 26 is utilized because changes in manifold pressure can be a good and convenient indicator of engine speed. Other sensors or engine operating parameters can also be utilized to provide the input required to modify speaker 22 outputs to match engine performance.

Referring to FIG. 4, the method steps utilized to synchronize the sound generated from the speaker 22 with engine performance are shown. A signal received from the MAP sensor 26 is utilized to estimate engine speed and throttle position. The MAP sensor 26 provides an estimate of engine speed from pressure frequency spectrum and an estimate of throttle position by measuring the mean vacuum pressure within the manifold 17. The engine speed and throttle position are indicative of current engine operating conditions. The desired sound emitted from the engine is tailored to correspond to the current engine operating conditions. For example, high engine speed corresponds to a louder, sound that conveys to the impression of power.

Changes in engine operating conditions are detected by the MAP sensor 26 and communicated to the controller 24. As appreciated, the controller 24 can be a stand-alone application specific controller for driving the speaker 22 of this system 14, or a portion of a vehicle controller that includes portions that command various functions of the vehicle 10.

The controller 24 utilizes data indicative of engine speed to generate signals to the speaker 22 that will generate the desired engine tones 30. Based on the engine speed and the desirable acoustic character, the controller 24 will generate a multitude of tones. The tones that are generated are all higher harmonics of the engine half order. Each tone generated by the speaker 22 is essentially a harmonic wave of an appropriate magnitude. The phase of each tone is scattered to avoid undesirable behavior of the signals that drive the speaker 22 and an excessive load on the the speaker 22.

The generated tones 30 are then compensated for according to a vehicle transfer function as indicated at 32. This is accomplished according to a time-domain, where a recursive or non-recursive digital filter is applied to the output. The digital filter represents an inverse of the vehicle transfer function. Alternatively this can be done in a frequency domain where the frequency response is broken down into magnitude and phase and subsequently applied to each of the tones. The final output signal 34 is then forwarded to the speaker 22 to produce the desired sound.

The sound emitted from the speaker 22 is separate from those sounds emitted by the engine 12. The sound is predetermined based on a calibration of engine speeds and throttle values. The acoustic performance is varied for each engine operating condition and a specific frequency and tone is selected based on the desired resultant sound that is emitted from the engine 12. The quality and tone of the desired sound is determined based on psycho-acoustic values assigned by a vehicle operator. The sound is tailored to engine operating conditions to communicate the desirable sounds of a powerful engine. Once the desired sounds are determined, the sound characteristics are stored within the controller 24 and correlated to engine operating conditions.

In operation, the MAP sensor 26 communicates data indicative of engine operation to the controller 24. Preferably, the MAP sensor 26 communicates data indicative of engine speed and throttle position. The controller 24 correlates engine speed and throttle position to the predetermined sound and generates a signal to drive the speaker 22. The speaker 22 generates the tones to provide the desired engine sounds. The actual sound produced by the engine 12 is not accounted for; instead the predetermined correlation to engine speed is utilized to simplify controller operation such that noise sensing devices and the accompanying control methods are not required.

The controller 24 also provide for the customization of engine sound depending on a user-selected profile. A user-selected profile can be engaged by a switch 36 (FIG. 1) or through instructions that are part of the controller programming. The user-selected profile can include, for example, changes between aggressive and passive engine noises. Each user-selected profile is tailored to produce a set of desirable engine noises responsive to engine operating conditions. In some instances, it may be desirable to emit a less aggressive noise form the engine. While in other cases the operator may desire that the engine covey the additional power produced by the increase airflow 28 provided by the unobstructed air passage 15.

The controller 24 and speaker 22 tailor sound to provide optimal performance within the limits of the controller electronics. The controller 24 includes an electronic package including an electronic board having an amplifier and a microprocessor. The electronic board, amplifier and microprocessor are all of a known configuration that is commercially available. A worker with the benefit of this disclosure would understand how to program a commercially available microprocessor and amplifier to drive the speaker 22 and produce the desired sounds responsive to a detected engine speed.

The controller 24 is preferably of a simple construction that requires few inputs. Preferably, inputs to the controller 24 include power connections to a power source within the vehicle 10 and a connection to sensor providing information indicative of engine performance. The minimal connections make the air induction system 14 highly adaptable to installation on vehicle of varying configurations. Further, the simplified connections and construction of the duct 16 make this system 14 desirable for after market applications.

The air induction system 14 of this invention provides an active noise control device suited for any vehicle engine. The duct 16 provides for greatly improved airflow that enhances engine performance. The speaker 22 produces tones that counter the undesirable side-affects produced by the unobstructed passage 15 to produce desirable engine sounds that are tailored to engine operating conditions and reflect the improved engine performance.

Although a preferred embodiment of this invention has been disclosed, a worker of ordinary skill in this art would recognize that certain modifications would come within the scope of this invention. For that reason, the following claims should be studied to determine the true scope and content of this invention.

What is claimed is:

1. An air induction system for a vehicle engine comprising:
a duct defining a passage for airflow;
a speaker for generating sound; and
a controller for driving said speaker to generate a desired sound responsive to engine operating conditions.

2. The system as recited in claim 1, wherein said controller drives said speaker to generate a desired sound responsive to engine speed.

3. The system as recited in claim 1, wherein said controller drives said speaker to generate multiple tones responsive to current engine operating conditions.

4. The system as recited in claim 3, comprising a sensor communicating information indicative of engine operating conditions to said controller.

5. The system as recited in claim 4, wherein said sensor comprises a manifold absolute pressure sensor.

6. The system as recited in claim 1, wherein said controller drives said speaker to generate a desired sound according to a pre-selected sound profile.

7. The system as recited in claim 1, wherein said controller drives said speaker to generate tones of a higher harmonic than the engine half order.

8. The system as recited in claim 1, comprising a speaker chamber disposed adjacent said duct for supporting said speaker.

9. The system as recited in claim 8, wherein said speaker chamber is not in airflow communication with said passage.

10. The system as recited in claim 8, wherein said speaker chamber amplifies sound generated from said speaker.

11. The system as recited in claim 8, wherein said speaker chamber is integrally formed within said duct.

12. The system as recited in claim 1, comprising an air filter disposed within said duct wherein said speaker is disposed adjacent said air filter.

13. The system as recited in claim 1, comprising an air filter having a low resistance to airflow through said passage.

14. The system as recited in claim 1, wherein said passage comprises a cross-section absent of obstructions to the flow of air.

15. The system as recited in claim 1, wherein said passage does not include a resonator.

16. A method of improving air intake airflow and acoustic performance comprising the steps of:
a) removing substantially all restrictions to airflow within an airflow passage;
b) monitoring engine operating conditions; and
c) generating a sound responsive to the engine operating conditions to provide a desired overall sound.

17. The method as recited in claim 16, wherein said step b) comprises monitoring a pressure within an intake manifold.

18. The method as recited in claim 16, wherein said step c) comprises generating a sound responsive to engine speed.

19. The method as recited in claim 16, comprising the step of selecting a desired sound corresponding to an engine operating condition and generating said selected desired sound responsive to detecting the engine operating condition.

20. The method as recited in claim 16, comprising the step of generating said sound according to one of several user-selected modes.
